(19) United States
(12) Patent Application Publication
(10) Pub. No.: US 2019/0298643 A1
(43) Pub. Date: Oct. 3, 2019

(54) POLYMERIC SUNSCREEN MATERIALS

(71) Applicant: ARIZONA BOARD OF REGENTS ON BEHALF OF THE UNIVERSITY OF ARIZONA, Tucson, AZ (US)

(72) Inventors: Douglas A. Loy, Tucson, AZ (US); Nanayakkarawasan Pallage Ravindu Nanayakkara, Tucson, AZ (US); Robb E. Bagge, Tucson, AZ (US)

(21) Appl. No.: 16/317,495

(22) PCT Filed: Jul. 11, 2017
(86) PCT No.: PCT/US17/41564

§ 371 Date: Jan. 11, 2019

Related U.S. Application Data
(60) Provisional application No. 62/360,676, filed on Jul. 11, 2016.
(60) Provisional application No. 62/360,730, filed on Jul. 11, 2016.
(60) Provisional application No. 62/360,776, filed on Jul. 11, 2016.

(51) Int. Cl. A61K 8/84; A61K 8/04; A61K 8/06; A61K 8/02; A61Q 17/04

(52) U.S. Cl. CPC A61K 8/84; A61K 8/042; A61K 8/044; A61K 8/046; A61K 2800/412; A61K 8/0241; A61Q 17/04; A61K 2800/652; A61K 8/06

(57) ABSTRACT

Polymeric sunscreen particles are prepared from organic monomers such as aldehydes, phenols, amines and carboxylic acids. Polymerization methods include, oxidative polymerization, micro-emulsion polymerization and nucleation-directed polymerization. The particles have the ability to absorb both UVA and UVB radiation. The sunscreens prepared in the present invention are inexpensive and non-toxic and may be prepared from bio-based materials.

### GOVERNMENT SUPPORT

This invention was made with government support under Grant No. 1241783 awarded by NSF. The government has certain rights in the invention.

### FIELD OF THE INVENTION

The present invention relates to novel sunscreen agents which have the ability to absorb both UV-A and UV-B wavelength radiation. The sunscreens prepared in the present invention are inexpensive and nontoxic polymer-based particles which may be made from bio-based materials.

### BACKGROUND OF THE INVENTION

UV radiation (100-400 nm) represents ˜10% of electromagnetic radiation (sunlight) that reaches the Earth's surface, and can be separated into three main types: UVC (100-290 nm), which has the shortest wavelength and highest energy; UVB (290-320 nm); and UVA (320-400 nm), which has the longest wavelength and lowest energy. UVA is further divided into UVA-I (340-400 nm) and UVA-II (320-340 nm). In small doses, UV radiation can be beneficial for vitamin D production or as therapeutic treatment for skin disorders, but too much exposure can cause several harmful photo-biological effects such as erythema (sunburn); accelerated skin ageing resulting in a variety of visible effects such as pronounced deep furrows, sagging, wrinkles, uneven pigmentation, dryness, and a leathery appearance; and most alarming, skin cancer. In the case of the latter, exposure accounts for the development of approximately 90% of all non-melanoma carcinomas according to the Environmental Protection Agency (EPA), with an estimated 3.5 million+ new cases of basal and squamous cell carcinoma diagnosed in the United States each year.

UVB has traditionally been thought to be the most harmful radiation to skin, and the prevalence of UVB absorbing active ingredients in commercial sunscreens has reflected this belief. Recent studies, however, have detailed the harmful effects of UVA exposure as well, for sufficient doses of UVA, particularly UVA-II, can in fact induce sunburn. While the sunburn potential of UVA is less than that of UVB, exposure to lower energy UVA over a prolonged period will result in the same degree of sunburn as exposure to higher energy UVB for a shorter period of time.

Alterations to the elastic fibers, termed elastosis, is characterized by hyperplasia (increase in cell proliferation), causing the fibers to thicken, degrade and accumulate. Eventually, they degenerate into an amorphous mass and become granular in appearance. Substantial loss of collagen is also seen in response to extensive UV exposure, for it is replaced by the accumulated fibers from elastosis. Elastosis can be induced by both UVB and UVA radiation. Due to the deeper penetration depth of UVA in skin), increased absorption of UVA in persons protected with sunscreens that only filter UVB, and in turn extended hours in the sun without the warning of sunburn, UVA has been found to be the major contributor of accelerated skin aging. For this reason, commercial sunscreen formulations are increasingly including active ingredients to attenuate UVA radiation.

To mitigate UV exposure, the use of sunscreens, which contain active ingredients that block UV, is recommended. Organic absorber sunscreens provide protection by preventing the penetration of UV radiation into skin via absorption of high energy UV, at which point they undergo transitions from the ground state to a higher energy state that correspond to the energy of radiation absorbed. This absorption is mediated by emission of lower energy radiation in the form of isomerization, visible light (fluorescence), or infrared (heat) back to the ground state. Compounds that fit these criteria are divided into several structural classes including p-aminobenzoic acid and analogues (PABAs), salicylates, cinnamates, benzophenone, dibenzoylmethanes, and camphor derivatives among others. While these examples are successful at absorbing UV radiation, they are limited in safety and effectiveness over time due to UV-induced decomposition and photo-toxicity, as well as photo-allergenic effects. For example, salicylates are UVB absorbing sunscreens that can cause allergic reactions and skin inflammation. Sunscreen compounds are even known to systemically absorb into the body after topical application, leading to concerns about their estrogenicity. As a result, stabilization and isolation from skin has increasingly become necessary to ensure their continual use.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

It is an objective of the present invention to provide for sunscreen compositions that have the ability to strongly absorb both UVA and UVB radiation and are nontoxic. Embodiments of the invention are given in the independent and dependent claims. Embodiments of the present invention can be freely combined with each other if they are not mutually exclusive.

According to some embodiments, the sunscreen composition is formed by copolymerization of an aldehyde and a phenolic aromatic. In some embodiments, the aldehyde is formaldehyde. In other embodiments, the aldehyde is an aromatic and conjugated aldehyde.

According to some embodiments, the sunscreen composition is formed by an oxidative polymerization of dopamine. In some embodiments, caffeic acid is introduced in the oxidative polymerization step of dopamine. The UV absorption property of the present sunscreen can be engineered to shorter wavelength by introducing caffeic acid into the oxidative polymerization step. The resulting polymer generates synthetic eumelanin particles which can be used as a new class of sunscreens.

In some embodiments, the sunscreen composition comprises spherical polymeric particles. According to some embodiments, the polymeric particles are thermoset. In some embodiments, the sunscreen composition is a suspension or lotion.

One of the unique and inventive technical features of the present invention is the use of copolymers to form polymeric sunscreen particles. Without wishing to limit the invention to any theory or mechanism, it is believed that the technical feature of the present invention advantageously provides for the ability to tune both the visible and UV absorption of the particles. None of the presently known prior references or work has the unique inventive technical feature of the present invention

### DESCRIPTION OF PREFERRED EMBODIMENTS

As used herein, the term “oxidative polymerization” refers to a polymerization wherein one or more monomers is oxidized. As used herein, the term “oxidatively polymerized” refers to a polymer which was formed by oxidative polymerization.

As used herein, the term “micro-emulsion” refers to a thermodynamically stable isotropic dispersion of water, oil and surfactants. As used herein, the term “micro-emulsion polymerization” refers to a polymerization which takes place within a micro-emulsion and generates polymeric particles.

As used herein, the term “nucleating species” refers aggregating molecules which form a separate phase. As used herein, the term “nucleation-directed polymerization” refers to a polymerization in which nucleating species grow via polymerization to form polymeric particles.

According to one embodiment, the present invention features a sunscreen composition. In some embodiments, the sunscreen composition comprises a polymeric particle. In one embodiment, the particle may be formed by the reaction of a phenolic compound and an aldehydic compound. In preferred embodiments, the particle is capable of absorbing UV radiation.

In another embodiment, the present invention features a method of forming a UV absorbing polymeric particle. As a non-limiting example, the method may comprise providing a phenolic compound, providing an aldehydic compound, and reacting the phenolic compound with the aldehyde compound to form said polymeric particle, wherein the particle is capable of absorbing UV radiation.

In some embodiments, the aldehydic compound may be formaldehyde. In other embodiments, the aldehydic compound may be aromatic or conjugated. For a non-limiting example, the aldehydic compound may be cinnamaldehyde or benzaldehyde. In further embodiments, the aldehydic component may be according to one or more of the following structures:

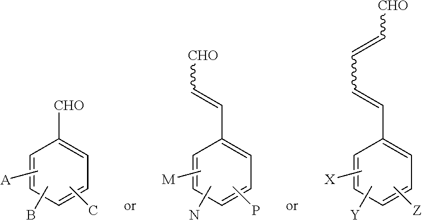

wherein A, B, C, M, N, P, X, Y and Z are each an H, CH₃, Cl, NO₂, F, Br, CN, CHO, CO₂Me, OMe, OH, Ph or PhCH═CH—.

In some embodiments, the phenolic component may be according to one or more of the following structures:

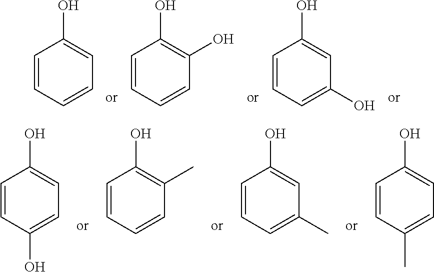

In preferred embodiments, the particle may be a thermoset particle. The particle may be spherical. In some embodiments the particle may be dispersed, suspended or dissolved in a liquid. For a non-limiting example, the liquid may be water, glycerol, an oil or an organic solvent. In further embodiments, the composition may be in the form of a dispersion, a suspension, an emulsion, a lotion or a liquid spray.

In other preferred embodiments, the particle may be configured to absorb both UVA and UVB radiation. In one embodiment, a color of the particle may be tuneable based on the structure and ratio of the phenolic component and the aldehydic component. For a non-limiting example, the particle may be various shades of red, tan, cream, brown or black in color.

According to one embodiment, the particle may be formed by a micro-emulsion polymerization or by a nucleation-directed polymerization. In some embodiments, the particle may be formed from one or more bio-based precursors such as resorcinol or cresol. In another embodiment, the particle may be nontoxic. In another embodiment, the particle may be formed in basic conditions. As a non-limiting example, the particle may be formed in the presence of ammonium hydroxide, sodium hydroxide or potassium hydroxide.

In a preferred embodiment, the particle may be about 0.1-100 microns in diameter. In other embodiments, the particle may be about 0.1-0.25, 0.25-0.5, 0.5-0.75, 0.75-1, 1-2.5, 2.5-5, 5-7.5, 7.5-10, 10-25, 25-50, 50-75 or 75-100 microns in diameter.

In preferred embodiments, the molar ratio of aldehydic compound to phenolic compound may be about 2:1. In other embodiments, the molar ratio of aldehydic compound to phenolic compound may be about 10:1, 5:1, 3:1, 1:1, 1:2, 1:3, 1:5, 1:10.

In some embodiments, the sunscreen composition may comprise polymeric particles according to at least one of the following structures:

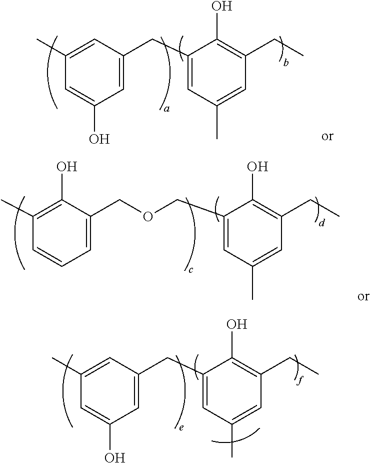

where a, b, c, d, e and f are each about 0-1,000,000.

In some embodiments, the sunscreen composition may comprise polymeric particles according to at least one of the following structures:

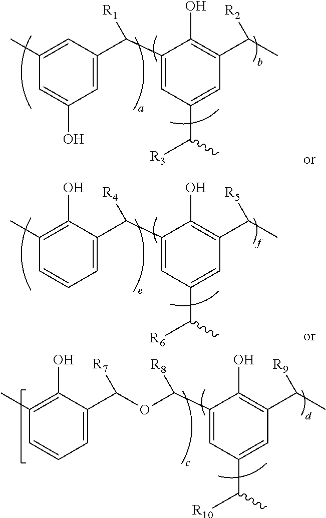

where a, b, c, d, e and f are each about 0-1,000,000, where R₁, R₂, R₃, R₄, R₅, R₆, R₇, R₈, R₉ and R₁₀ are each according to at least one of the following structures:

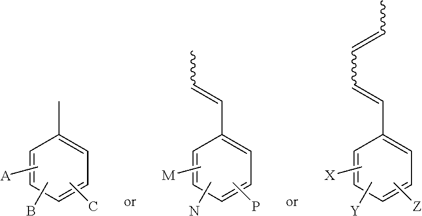

and where A, B, C, M, N, P, X, Y and Z may each be a H, CH₃, Cl, NO₂, F, Br, CN, CHO, CO₂Me, OMe, OH, Ph or PhCH═CH—.

In one embodiment, the present invention features a sunscreen composition, comprising a polymeric particle formed from an amine compound, wherein the amine compound is oxidatively polymerized to form said particle and the particle is capable of absorbing UV radiation.

In another embodiment, the present invention features a UV absorbing, polymeric particle, comprising providing an amine compound and oxidatively polymerizing the amine compound to form a polymer, wherein the polymer forms said particle and the particle is capable of absorbing UV radiation.

In preferred embodiments, the amine compound may be dopamine. In further embodiments, one or more co-monomers may be polymerized with the amine compound and the particle formed comprises a copolymer. In one embodiment, a color of the particle may be tuneable based on the structures and ratio of the amine component and the one or more co-monomers.

In some preferred embodiments, the co-monomer may absorb shorter wavelength UV radiation than the amine compound. According to one embodiment, at least one of the co-monomers may be an aromatic or conjugated carboxylic acid. As a non-limiting example, the carboxylic acid may be caffeic acid. In some embodiments, the carboxylic acid may be according to the following structure:

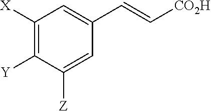

wherein X, Y and Z are each H, OH or Ome.

In one embodiment, the particle may be a thermoset particle. In another embodiment, the particle may be configured to act as a synthetic eumelanin. In yet another embodiment, the particle may be configured to absorb both UVA and UVB radiation. In still another embodiment, the particle may be formed by a micro-emulsion polymerization or a nucleation-directed polymerization. In some embodiments, the particle may be non-toxic.

In a preferred embodiment, the particle may be about 0.1-100 microns in diameter. In other embodiments, the particle may be about 0.1-0.25, 0.25-0.5, 0.5-0.75, 0.75-1, 1-2.5, 2.5-5, 5-7.5, 7.5-10, 10-25, 25-50, 50-75 or 75-100 microns in diameter.

According to some embodiments, the present invention may feature a sunscreen formulation comprising any of the sunscreen compositions described herein, in an amount effective to absorb UV radiation, and a cosmeceutically-acceptable sunscreen carrier comprising at least one solubilizer, and at least one cosmetic adjuvant. As a non-limiting example, the adjuvant may be a preservative, antifoam, perfume, oil, wax, propellant, dye, pigment, film-former or waterproofing agent, emulsifier, surfactant, thickener, humectant, binder, exfoliant or emollient.

According to one embodiment, the formulation may be in a form suitable for topical application. Non-limiting examples include creams, ointment, suspensions, powders, lotions, gels, solids, foams, emulsions, liquid dispersions, sprays and aerosols.

In another embodiment, the present invention may feature a sunscreen formulation comprising any of the sunscreen compositions described herein, in an amount effective to absorb UV radiation, and a pharmaceutically-acceptable sunscreen carrier.

In still another embodiment, the present method may feature a method of protecting a skin of a mammal from harmful photo-biological effects of UV radiation. The method may comprise topically applying to the skin of the mammal an effective coating of any of the sunscreen formulations described herein.

Scheme 1. Non-limiting example of a reaction schematic for a copolymerization of formaldehyde and resorcinol to form polymeric particles.

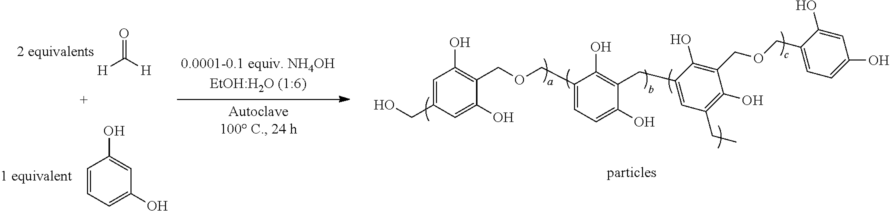

where a, b and c are each 1-100,000.

Scheme 2: Non-limiting example of a reaction schematic for a copolymerization of formaldehyde and cresol to form polymeric particles.

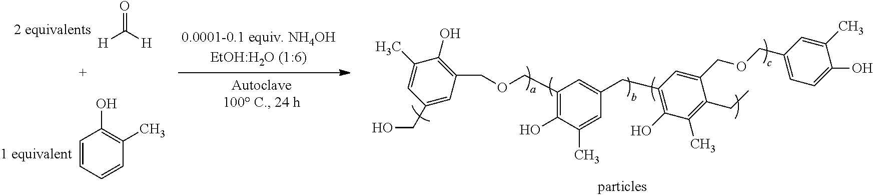

where a, b and c are each 1-100,000.

Scheme 3: Non-limiting example of a reaction schematic for a copolymerization of resorcinol and an aldehydic compound to form polymeric particles.

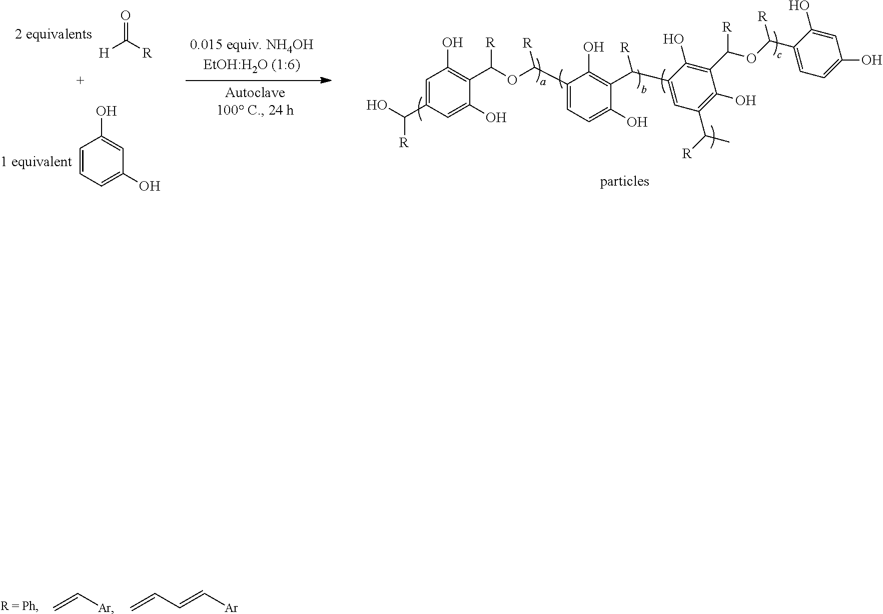

where a, b and c are each 1-100,000.

The following are exemplary synthetic procedures, and are included here as non-limiting examples only. Equivalents or substitutes are within the scope of the invention.

### Example 1: Formation of Polymeric Particles from Resorcinol and Formaldehyde

Monodisperse RF resins spheres were synthesized by using resorcinol and formaldehyde solution as precursors. A 0.1 mL volume of ammonia aqueous solution (NH₄OH, 29 wt %) was mixed with a solution containing 8 mL of absolute ethanol (EtOH) and 20 mL of deionized water (H2O) (with total amount of 28 mL) to prepare RF resins spheres.

After stirring for more than 1 h, a volume of 0.28 mL formaldehyde solution and 0.2 g of resorcinol was added to each of the reaction solution and stirred for 24 h at 30° C., and subsequently heated for 24 h at 100° C. under a static condition in a Teflon-lined autoclave. The solid product was recovered by centrifugation and air-dried at 100° C. for 48 h.

### Example 2: Formation of Polymeric Particles from Resorcinol and Cinnamaldehyde

Monodisperse resorcinol-cinnamaldehyde resin particles were synthesized by using resorcinol and cinnamaldehyde solution as precursors. A 0.1 mL volume of ammonia aqueous solution (NH4OH, 29 wt %) was mixed with a solution containing 8 mL of absolute ethanol (EtOH) and 20 mL of deionized water (H2O) (with totally amount of 28 mL) to prepare RC resins spheres. After stirring for more than 1 h, a volume of 0.46 mL Cinnamaldehyde solution and 0.2 g of resorcinol was added to each of the reaction solution and stirred for 24 h at 30° C., and subsequently heated for 24 h at 100° C. under a static condition in a Teflon-lined autoclave. The solid product was recovered by centrifugation and air-dried at 100° C. for 48 h.

### Example 3: Formation of Polymeric Particles from Resorcinol and Benzaldehyde

Monodisperse Resorcinol-benzaldehyde resin particles were synthesized by using resorcinol and benzaldehyde solution as precursors. A 0.1 mL volume of ammonia aqueous solution (NH4OH, 29 wt %) was mixed with a solution containing 8 mL of absolute ethanol (EtOH) and 20 mL of deionized water (H2O) (with totally amount of 28 mL) to prepare RC resins spheres. After stirring for more than 1 h, a volume of 0.38 mL benzaldehyde solution and 0.2 g of resorcinol was added to each of the reaction solution and stirred for 24 h at 30° C., and subsequently heated for 24 h at 100° C. under a static condition in a Teflon-lined autoclave. The solid product was recovered by centrifugation and air-dried at 100° C. for 48 h.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

1. A sunscreen composition, comprising a polymeric particle formed from;
a. a phenolic compound; and
b. an aldehydic compound;
wherein the phenolic compound reacts with the aldehydic compound to form said particle; and wherein said particle is capable of absorbing UV radiation.

2. (canceled)

3. The composition of claim 1, wherein the aldehydic compound is aromatic or conjugated.

4.-6. (canceled)

7. The composition of claim 1, wherein the particle is a thermoset particle.

8. The composition of claim 1, wherein the composition is in the form of a dispersion, a suspension, an emulsion, a lotion or a liquid spray.

9. The composition of claim 1, wherein the particle is configured to absorb both UVA and UVB radiation.

10. The composition of claim 1, wherein a color of the particle is tuneable based on the structures and ratio of the phenolic component and the aldehydic component.

11. The composition of claim 1, wherein the particle is formed by a micro-emulsion polymerization or by a nucleation-directed polymerization.

12. The composition of claim 1, wherein the particle is formed in basic conditions.

13. The composition of claim 1, wherein the particle is nontoxic.

14. The composition of claim 1, wherein the particle is about 0.1-100 microns in diameter.

15.-29. (canceled)

30. A sunscreen composition, comprising a polymeric particle formed from an amine compound;
wherein the amine compound is oxidatively polymerized to form said particle, and
wherein the particle is capable of absorbing UV radiation.

31. (canceled)

32. The composition of claim 30, wherein one or more co-monomers are polymerized with the amine compound and the particle formed comprises a copolymer.

33. The composition of claim 32, wherein a color of the particle is tuneable based on the structures and ratio of the amine component and the one or more co-monomers.

34. The composition of claim 32, wherein at least one of the co-monomers is an aromatic or conjugated carboxylic acid.

35.-36. (canceled)

37. The composition of claim 32, wherein the co-monomer absorbs shorter wavelength UV radiation than the amine compound.

38. The composition of claim 30, wherein the particle is a thermoset particle.

39. The composition of claim 30, wherein the particle is configured to act as a synthetic eumelanin.

40. The composition of claim 30, wherein the composition is in the form of a dispersion, a suspension, an emulsion, a lotion or a liquid spray.

41. The composition of claim 30, wherein the particle is configured to absorb both UVA and UVB radiation.

42. The composition of claim 30, wherein the particle is formed by a micro-emulsion polymerization or by a nucleation-directed polymerization.

43.-62. (canceled)
